(19) United States
(12) Patent Application Publication
(10) Pub. No.: US 2026/0090541 A1
(43) Pub. Date: Apr. 2, 2026

(54) TARGETED NANO-CARRIER, PREPARATION METHOD THEREFOR, APPLICATION THEREOF, TARGETED DRUG-LOADED NANO-CARRIER, AND PREPARATION METHOD THEREFOR

(71) Applicant: HENAN UNIVERSITY, Henan (CN)

(72) Inventors: Bingyang SHI, Henan (CN); Xue XIA, Henan (CN); Meng ZHENG, Henan (CN); Yang LIU, Henan (CN); Aijie LI, Henan (CN)

(21) Appl. No.: 19/110,304

(22) PCT Filed: Sep. 8, 2023
(86) PCT No.: PCT/CN2023/117689

§ 371 Date: Mar. 10, 2025

(30) Foreign Application Priority Data
Sep. 8, 2022 (CN) 202211094750.0

(51) Int. Cl. A01N 25/10; A01N 25/04; B82Y 5/00

(52) U.S. Cl. CPC A01N 25/10; A01N 25/04; B82Y 5/00

(57) ABSTRACT

The present disclosure belongs to the technical field of functional materials, and provided are a targeted nano-carrier, a preparation method therefor, an application thereof, a targeted drug-loaded nano-carrier, and a preparation method therefor. The targeted nano-carrier comprises a nano-carrier and a target chemically bonded on the nano-carrier, wherein the nano-carrier is nano-particles formed from an organic polymer or an inorganic material, and the target is aspartic acid or an aspartic acid derivative. The targeted nano-carrier provided by the invention can actively penetrate plant cell walls and cell membranes, is suitable for drug delivery for living plants, tissues etc., can reduce drug dosage and costs, has a protective effect on carried drugs and improves drug efficiency, prolongs the duration of drug action, reduces toxicity and contamination, and reduces the likelihood of drug resistance.

### CROSS-REFERENCE TO RELATED APPLICATION

The present disclosure claims the priority to the Chinese patent application with the filing No. 202211094750.0 filed with the Chinese Patent Office on Sep. 8, 2022, and entitled “TARGETED NANO-CARRIER, PREPARATION METHOD THEREFOR, APPLICATION THEREOF, TARGETED DRUG-LOADED NANO-CARRIER, AND PREPARATION METHOD THEREFOR”, the contents of which are incorporated herein by reference in entirety.

### TECHNICAL FIELD

The present disclosure relates to the technical field of functional materials, and particularly to a targeted nano-carrier, a preparation method therefor, use thereof, a targeted drug-loaded nano-carrier and a preparation method therefor.

### BACKGROUND ART

Scientists utilize plant biotechnology for high-yield and stress-resistant crop screening, improved drug biosynthesis, sustainable agriculture development, and the like. However, even after decades of progress, the application of biotechnology in plant science and even agricultural production still face a variety of problems and challenges. Cell walls in plant cells constitute a major barrier for delivering exogenous biological macromolecules and other drugs. During molecular breeding optimization through plant genetic transformation, a conventional gene gun has shortcomings such as damage of tissue of interest and low gene expression level, while the Agrobacterium transformation method struggles with narrow host selectivity and tissue specificity. Moreover, callus generation required by these methods also restricts applicable plant species. Regarding conventional physical and chemical pesticide and fertilizer applications, excessive application and high residual due to extremely low utilization rates poses enormous pressure on production safety and environment.

Nanodelivery carriers have been widely employed in the biomedicine field, making substantial contributions to human health. Nanodelivery can dramatically reduce drug dosage through targeted aggregation only in required tissues, thereby lowering medication costs, enhancing drug efficacy, prolonging therapeutic duration, reducing toxicity and contamination, and decreasing drug resistance probability. However, there are few nanodelivery carriers currently used for plants due to the natural barrier formed by cell walls in plant systems, which prevents almost all nano-carriers from effectively penetrating the cell walls for delivery into living tissues and cells. Limited researches on plant nanodelivery carriers also required external assistance (such as leaf injection and magnetic force) to achieve their goal of penetrating cell walls into living plant tissues. However, applications in plant biotechnology, especially in agricultural production, typically demand a huge sample size, and even laboratory operations often require hundreds of samples. Therefore, any method requiring external assistance is cumbersome and inefficient. Therefore, it is a technical problem to be solved urgently to develop nanodelivery carriers capable of actively penetrating plant cell walls and further cell membranes, without requiring external assistance or causing tissue damage, enabling easy application to a large number of plants, and delivering drugs to plants.

### SUMMARY

The present disclosure aims at providing a targeted nano-carrier and a preparation method therefor and use thereof, and a targeted drug-loaded nano-carrier and a preparation method therefor. The targeted nano-carrier provided by the present disclosure can actively penetrate plant cell walls and cell membranes, and is suitable for delivering drugs to plants.

In order to achieve the above objective, the present disclosure provides technical solutions as follows.

The present disclosure provides a targeted nano-carrier, including a nano-carrier and a target chemically bonded on the nano-carrier, where the nano-carrier is a nano-particle formed from an organic polymer or an inorganic material, and the target is aspartic acid or an aspartic acid derivative.

Optionally, a targeting group provided by the aspartic acid has any of structures shown in Formulas I-IV:

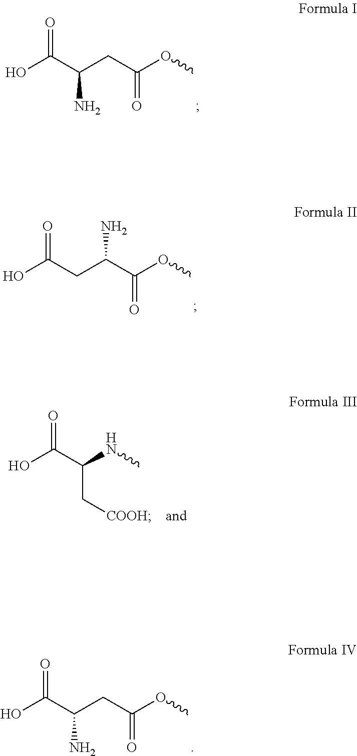

Optionally, the nano-carrier has a particle size of 10-1000 nm.

Optionally, a number-average molecular weight of the organic polymer is 3-50 kDa.

Optionally, the organic polymer includes a hydrophobic polymer and a hydrophilic linker covalently linked to the hydrophobic polymer, and the hydrophilic linker is chemically bonded to the target.

The present disclosure provides a preparation method for the targeted nano-carrier in the above technical solutions, including a following step:
modifying the target on the nano-carrier via chemical bonding in the presence of a solvent, so as to yield the targeted nano-carrier.

The present disclosure provides use of the targeted nano-carrier in the above technical solutions or the targeted nano-carrier prepared by the preparation method in the above technical solution as an active targeted nano-carrier for living plants, tissues of living plants, organs of living plants, cells of living plants, in vitro cultured explants, in vitro cultured callus, in vitro cultured plant tissues or in vitro cultured plant cells.

The present disclosure provides use of the targeted nano-carrier in the above technical solutions or the targeted nano-carrier prepared by the preparation method in the above technical solution as an active targeted Nanodelivery carrier for living plants.

The present disclosure provides a targeted drug-loaded nano-carrier, including a targeted nano-carrier and a drug encapsulated in the targeted nano-carrier, where the targeted nano-carrier is the targeted nano-carrier in the above technical solutions or the targeted nano-carrier prepared by the preparation method in the above technical solution.

Optionally, the drug includes a small molecule drug or a biomacromolecule, and drug-loading rate of the targeted drug-loaded nano-carrier is 1-99%.

The present disclosure provides a preparation method for the targeted drug-loaded nano-carrier in the above technical solutions, including a following step:
mixing the targeted nano-carrier, a drug and a solvent, and subjecting the mixture to an encapsulation processing, yielding the targeted drug-loaded nano-carrier.

The present disclosure provides a targeted nano-carrier, including a nano-carrier and a target chemically bonded on the nano-carrier, where the nano-carrier is a nanoparticle formed from an organic polymer or an inorganic material, and the target is aspartic acid or an aspartic acid derivative. The targeted nano-carrier provided by the present disclosure can actively penetrate plant cell walls and cell membranes, is suitable for drug delivery for living plants, tissues etc., can reduce drug dosage and costs, have a protective effect on loaded drugs and improve drug efficacy, prolong therapeutic duration, reduce toxicity and contamination, and reduce drug resistance probability.

### BRIEF DESCRIPTION OF DRAWINGS

FIG. 1 is a flowchart of preparing Asp-NP and an application schematic diagram of a targeted drug-loaded nano-carrier as a drought-resistant agent obtained by loading ABA as an example;

FIG. 2 is a nuclear magnetic resonance spectrogram of Asp-PEG-PDPA with L-Asp as a targeting group prepared in Example 1;

FIG. 3 is a transmission electron microscope image of L-Asp-NP in Example 1;

FIG. 4 shows comparison graphs of particle size and drug-loading rate of Asp-NP@ABA (specifically, D-Asp-NP@ABA, A-Asp-NP@ABA, N-Asp-NP@ABA and L-Asp-NP@ABA) obtained after loading ABA and non-targeted NP@ABA as control;

FIG. 5 shows confocal laser microscope images of DiO tracking result at a 20-μm depth in Arabidopsis leaves, observed 36 h after the leaves were sprayed with different treatments in Application Example 1;

FIG. 6 is a depth statistical chart of DiO penetrating Arabidopsis leaf tissues brought by nanoparticles tracked by the confocal laser microscope, 36 h after the leaves were sprayed with different treatments in Application Example 1;

FIG. 7 is a comparison graph of DiO signals brought into protoplasts by the nanoparticles tracked by the confocal laser microscope, where different treatments in Application Example 1 and isolated Arabidopsis mesophyll protoplast were co-cultured for 4 h, followed by replacement with fresh MS (Murashige and Skoog) medium and culturing for 20 h;

FIG. 8 shows confocal laser microscope images of FITC tracking results at various depths of Commelina communis leaves, observed 6 h after the leaves were sprayed with different treatments in Application Example 1;

FIG. 9 is a comparison graph of germination ratios of Arabidopsis seeds with different treatments;

FIG. 10 shows comparison images of different penetration depths of Asp-NP-FITC into Arabidopsis root tissues at different time points;

FIG. 11 shows comparison images of different penetration depths of Asp-NP-FITC into soybean root tissues after 4 h and 6 h;

FIG. 12 shows comparison images of different penetration depths of Asp-NP-FITC into maize root tissues after 4 h and 6 h;

FIG. 13 is a comparison chart of leaf senescence and yellowing in Arabidopsis seedlings induced by root-absorbed targeted nano-carrier with different treatments in a hydroponic system;

FIG. 14 shows comparison images of extended survival periods of Arabidopsis seedlings under water withholding conditions after the seedlings were sprayed with different treatments in Application Example 3;

FIG. 15 is a comparison chart of survival rates of Arabidopsis seedlings after being sprayed with different treatments in Application Example 3;

FIG. 16 is a percent scatter plot of extended survival periods of Arabidopsis seedlings under water withholding conditions after the seedlings were sprayed with different treatments in Application Example 3, relative to the extended survival period in the MS treatment group;

FIG. 17 shows results of minimum effective concentrations of Asp-NP@ABA determined with survival period under water withholding conditions as a criterion in Application Example 3 (with outcomes of ABA treatment as references);

FIG. 18 is a comparison chart of ABA content in protoplast and apoplast of Arabidopsis leaves, 24 h after being sprayed with different treatments in Application Example 3;

FIG. 19 shows comparison images of extended survival periods of soybean seedlings under water withholding conditions after the seedlings were sprayed with different treatments in Application Example 4;

FIG. 20 is a median plot of survival values of extended survival periods of soybean seedlings under water withholding conditions after the seedlings were sprayed with different treatments in Application Example 4;

FIG. 21 is a comparison graph of extended survival periods of maize seedlings under water withholding conditions after the seedlings were sprayed with different treatments in Application Example 4;

FIG. 22 shows comparison graphs of Arabidopsis seed germination ratios under influence of drug-loaded products added with a non-targeted polymer (i.e., MeO-PEG-PDPA) at different ratios in Application Example 5; and

FIG. 23 shows comparison images of survival period extension of Arabidopsis under water withholding conditions under influence of the drug-loaded products added with the non-targeted polymer (i.e., MeO-PEG-PDPA) at different ratios in Application Example 5.

### DETAILED DESCRIPTION OF EMBODIMENTS

The present disclosure provides a targeted nano-carrier, including a nano-carrier and a target chemically bonded on the nano-carrier, where the nano-carrier is a nano-particle formed from an organic polymer or an inorganic material, and the target is aspartic acid or an aspartic acid derivative.

The targeted nano-carrier provided in the present disclosure includes the nano-carrier. The nano-carrier is a nano-particle formed from an organic polymer or an inorganic material. In the present disclosure, the nano-carrier optionally has a particle size of 10-1000 nm, and more optionally 20-200 nm.

In the present disclosure, a number-average molecular weight of the organic polymer is optionally 3-50 kDa, and more optionally 5-20 kDa. In the present disclosure, the organic polymer optionally includes a hydrophobic polymer and a hydrophilic linker covalently linked to the hydrophobic polymer, and the hydrophilic linker is chemically bonded to the target.

In the present disclosure, the hydrophobic polymer optionally includes any of substances involved in (1), (2) and (3) below:
(1) poly(lactic-co-glycolic acid) (PLGA), PLGA derivatives, polylactic acid (PLA), PLA derivatives, polycaprolactone (PCL), PCL derivatives, poly(methyl carbonate) (PMC), and PMC derivatives;
(2) one or more of glycolide, lactide, caprolactone and carbonate; and copolymers formed from at least two of glycolide, lactide, caprolactone and carbonate; and
(3) polyurethane (PU), PU derivatives, polyether ether ketone (PEEK), PEEK derivatives, polymethyl methacrylate (PMMA), PMMA derivatives, polyvinyl alcohol (PVA), PVA derivatives, polyethylene (PE), PE derivatives, hydrophobic polyamino acids, and hydrophobic polyamino acid derivatives, where the hydrophobic polyamino acid is optionally polyphenylalanine.

In the present disclosure, the hydrophilic linker optionally includes any of substances involved in (a) and (b) below:
(a) polyethylene glycol (PEG), polyethylene oxide (PEO), poly(ethylene glycol) methacrylate (POEG), poly 2-methacryloyloxyethyl phosphorylcholine (PMPC), polycarboxybetaine (PCB), dextran, dextran, hyaluronic acid, chitosan, β-cyclodextrin, hyperbranched polyglycidyl ether (HPG), poly N-(2-hydroxypropyl) methacrylamide (PHPMA), polyhydroxyethyl methacrylate (PHEMA), polyacrylamide (PAM), polyvinylpyrrolidone (PVP), polyacrylic acid (PAA), polymaleic anhydride (HPMA), and polyquaternary ammonium salts; and
(b) polyethyleneimine (PEI), PEI derivatives, pharmaceutically acceptable PEI salts, poly(N,N-dimethylaminoethyl methacrylate) (PDMAEMA), PDMAEMA derivatives, pharmaceutically acceptable PDMAEMA salts, polylysine (PLL), PLL derivatives, pharmaceutically acceptable PLL salts, hydrophilic polyamino acids, hydrophilic polyamino acid derivatives, and pharmaceutically acceptable hydrophilic polyamino acid salts, where the hydrophilic polyamino acid is optionally polyglutamic acid (PGu) or polyaspartic acid (PAsp).

In the present disclosure, when the nano-carrier is a nanoparticle formed from an organic polymer, morphology of the nano-carrier may be specifically micelle or vesicle, which is not particularly limited in the present disclosure.

In the present disclosure, the inorganic material optionally includes silicon, silicon oxides, iron, iron oxides, calcium, calcium oxides or carbon nanomaterials. In the present disclosure, specifically, the inorganic material contains an active group on a surface. In the present disclosure, optionally, the active group is directly chemically bonded to the target, or the active group is chemically bonded to the target via a hydrophilic linker, that is, the target may be chemically bonded to the hydrophilic linker first, and then on this basis, the hydrophilic linker is chemically bonded to the active group on the surface of the inorganic material, alternatively, the active group on the surface of the inorganic material may be chemically bonded to the hydrophilic linker first, and then on this basis, the hydrophilic linker is chemically bonded to the target. In the present disclosure, optional types of the hydrophilic linker are optionally consistent with those of the above hydrophilic linker, and will not be repeated herein. The present disclosure has no particular limitation on specific types of the active group, as long as the active group can be chemically bonded to the hydrophilic linker or the target, and specifically, the active group may be a hydroxyl group. Sources of the inorganic material are not particularly limited in the present disclosure, and any commercially available product well known to those skilled in the art can be used.

The targeted nano-carrier provided in the present disclosure includes the target, where the target is chemically bonded on the nano-carrier, and the target is aspartic acid or an aspartic acid derivative. In the present disclosure, the target may be chemically bonded to the nano-carrier specifically via an ester group or an amide group, which is not particularly limited in the present disclosure. In the present disclosure, a targeting group provided by the aspartic acid in the targeting nano-carrier optionally has any of structures shown in Formulas I-IV, designated as D-Asp, L-Asp-A, L-Asp-N, and L-Asp, respectively:

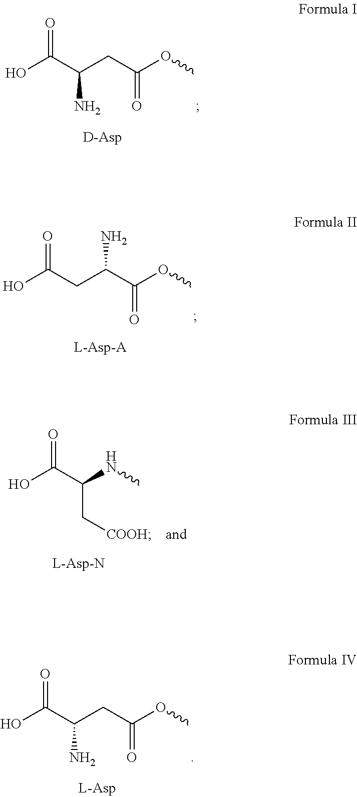

The present disclosure provides a preparation method for the targeted nano-carrier in the above technical solutions, including a following step:
modifying the target on the nano-carrier via chemical bonding in the presence of a solvent, so as to yield the targeted nano-carrier.

The present disclosure optionally selects a suitable method according to specific types of the nano-carrier and the target to chemically bond the two so as to prepare the targeted nano-carrier, which will be described in detail below.

In a first case, when the nano-carrier is a nanoparticle formed from an inorganic material, the present disclosure optionally chemically bonds the nano-carrier and the target through chemical reaction in the presence of a solvent, thereby yielding the targeted nano-carrier. The solvent and conditions of the chemical reaction are determined by types of the inorganic material and the target, and are not particularly limited in the present disclosure.

In a second case, when the nano-carrier is a nanoparticle formed from an organic polymer, according to properties of the organic polymer and the target, the present disclosure can prepare the targeted nano-carrier according to the above first case, that is, the nano-carrier is prepared first, and then the nano-carrier and the target are chemically bonded through chemical reaction in the presence of a solvent, thereby yielding the targeted nano-carrier, where the solvent and conditions of the chemical reaction are determined by types of the organic polymer and the target, and are not particularly limited in the present disclosure.

In a third case, when the nano-carrier is a nanoparticle formed from an organic polymer, according to properties of the organic polymer and the target, the present disclosure can further chemically bond the target on a monomer for preparing the organic polymer, and then realize the preparation of the targeted nano-carrier on the basis of the target-modified monomer, where a structure of the target-modified monomer and conditions for further preparing the targeted nano-carrier on this basis are determined by types of the organic polymer and the target, and are not particularly limited in the present disclosure.

In embodiments of the present disclosure, the preparation of the targeted nano-carrier (Asp-PEG-PDPA) using L-Asp as a targeting group, PDPA as a hydrophobic polymer, and PEG as a hydrophilic linker is taken as an example for illustration. In the present disclosure, the preparation method for Asp-PEG-PDPA with L-Asp as a targeting group optionally includes following steps:
mixing compound S1, compound S6, dicyclohexylcarbodiimide, 1-hydroxybenzotriazole, 4-dimethylaminopyridine and a first organic solvent, and subjecting the mixture to an amidation reaction, yielding compound S7;
mixing the compound S7, compound S4, azobisisobutyronitrile and a second organic solvent, and subjecting the mixture to a reversible addition-fragmentation chain transfer (RAFT) polymerization, yielding compound S8; and
mixing the compound S8, trifluoroacetic acid and a third organic solvent, and subjecting the mixture to a tert-butoxycarbonyl (Boc) removal reaction, yielding compound S9 (i.e., Asp-PEG-PDPA with L-Asp as a targeting group).

Structural formulae of the compound S1, compound S6, compound S7, compound S4, compound S8 and compound S9 are shown below:

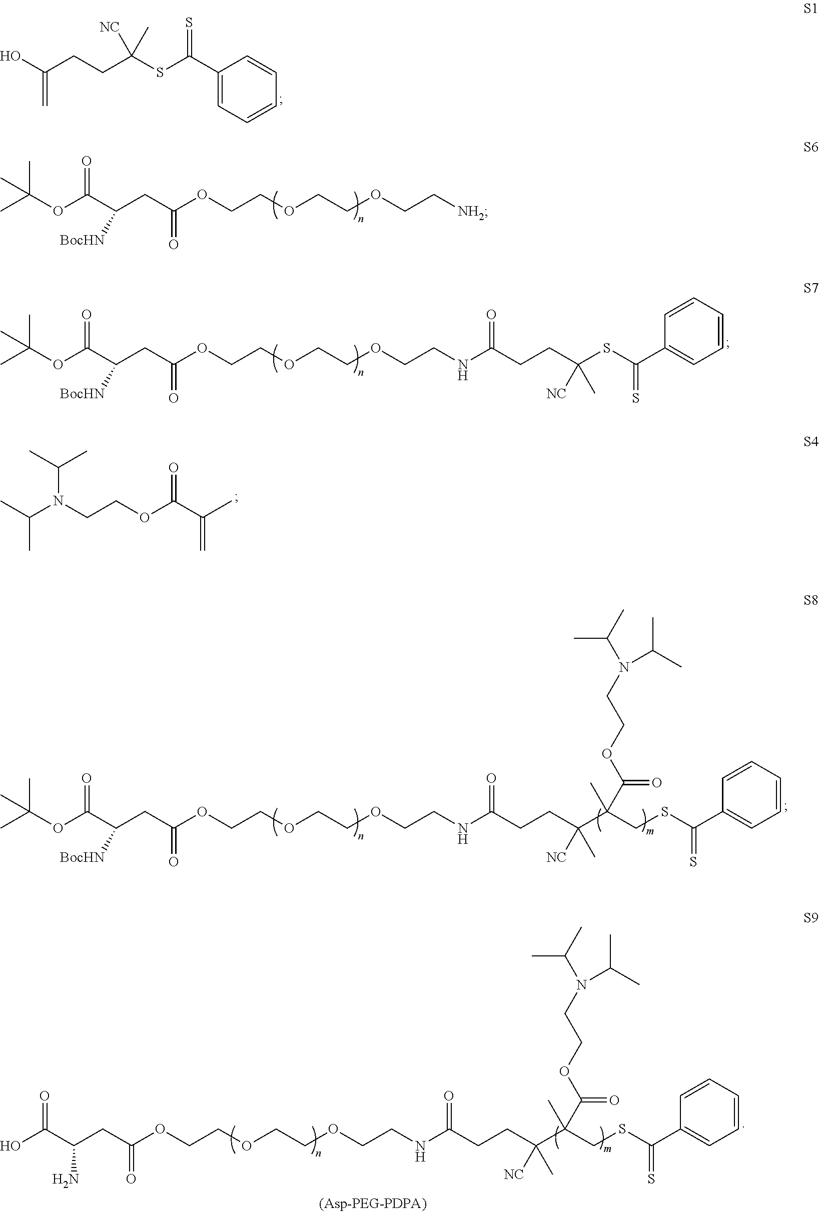

In the present disclosure, the compound S1, compound S6, dicyclohexylcarbodiimide, 1-hydroxybenzotriazole, 4-dimethylaminopyridine and the first organic solvent are mixed, and undergo the amidation reaction, yielding the compound S7. In the present disclosure, a molar ratio of the compound S1, compound S6, dicyclohexylcarbodiimide, 1-hydroxybenzotriazole and 4-dimethylaminopyridine is optionally 1:(0.2-3):(1-3):(1-3):(0.01-1), and more optionally 1:1:1.2:1.2:0.1; and the first organic solvent is optionally dichloromethane, and the present disclosure has no particular limitation to an amount of the first organic solvent, as long as the reaction can proceed smoothly. In the present disclosure, compound S1, dicyclohexylcarbodiimide, 1-hydroxybenzotriazole and 4-dimethylaminopyridine are optionally dissolved in the first organic solvent to render a mixed material; compound S6 is dissolved in the first organic solvent to render a compound S6 solution; and the compound S6 solution is added into the mixed material in one portion to undergo the amidation reaction. In the present disclosure, the amidation reaction is optionally carried out under a room-temperature condition, for optionally 4-48 h, and more optionally 24 h; and the amidation reaction is optionally carried out under a nitrogen protection condition. After the amidation reaction, in the present disclosure, the resulting product system is optionally subjected to rotary evaporation so as to remove the solvent, the resulting crude product is dissolved in ethyl acetate, and filtered to remove insoluble substances, and a filtrate is concentrated to yield the compound S7.

After the compound S7 is obtained, the present disclosure mixes the compound S7, compound S4, azobisisobutyronitrile and the second organic solvent, to undergo the RAFT polymerization, yielding compound S8. In the present disclosure, a molar ratio of the compound S4, compound S7 and azobisisobutyronitrile is optionally 25:(0.5-1.2):(0.01-0.5), and more optionally 25:1:0.1. The second organic solvent is optionally N,N-dimethylformamide, and the present disclosure has no particular limitation to an amount of the second organic solvent, as long as the reaction can proceed smoothly. In the present disclosure, compound S4 and azobisisobutyronitrile are optionally dissolved in the second organic solvent, and compound S7 is added into the resulting mixture under nitrogen protection for the RAFT polymerization. In the present disclosure, the RAFT polymerization is optionally conducted at a temperature of 40-100° C., more optionally 70° C., for optionally 4-48 h, and more optionally 24 h; and the RAFT polymerization is optionally conducted under the nitrogen protection condition. After the RAFT polymerization, in the present disclosure, the resulting product system is optionally cooled to room temperature, and then dialyzed in a dialysis bag, yielding the compound S8. In the present disclosure, a dialysate used in the dialysis is optionally anhydrous ethanol and ultrapure water in sequence, and dialysis periods using anhydrous ethanol and ultrapure water are optionally independently 6-72 h, and more optionally 24 h; and after the dialysis, in the present disclosure, a material in the dialysis bag is optionally collected and freeze-dried to yield the compound S8 as a pink amorphous solid.

After the compound S8 is obtained, in the present disclosure, the compound S8, trifluoroacetic acid and the third organic solvent are mixed, and undergo the tert-butoxycarbonyl removal reaction, yielding compound S9. In the present disclosure, a ratio of an amount of the compound S8 to an amount of trifluoroacetic acid is optionally 1 mmol:(10-1000) mL, and more optionally 1 mmol:100 mL; the third organic solvent is optionally dichloromethane, and the present disclosure has no particular limitation to the amount of the third organic solvent, as long as the reaction can proceed smoothly. In the present disclosure, the tert-butoxycarbonyl removal reaction is optionally conducted under a room-temperature condition for 0.5-48 h, and more optionally 24 h; and the tert-butoxycarbonyl removal reaction is optionally conducted under a nitrogen protection condition. After the tert-butoxycarbonyl removal reaction, in the present disclosure, the resulting product system is optionally subjected to rotary evaporation to remove the solvent and trifluoroacetic acid, and the resulting crude product is dissolved in ethyl acetate, and then dialyzed in a dialysis bag, yielding the compound S9. In the present disclosure, a dialysate used in the dialysis is optionally anhydrous ethanol and ultrapure water in sequence, and dialysis periods using anhydrous ethanol and ultrapure water are optionally independently 6-72 h, and more optionally 24 h; and after the dialysis, in the present disclosure, a material in the dialysis bag is optionally collected and freeze-dried to yield the compound S9 as a pink amorphous solid.

In the present disclosure, when D-Asp, L-Asp-A or L-Asp-N is used as a targeting group to prepare a corresponding targeted nano-carrier (PDPA is a hydrophobic polymer, PEG is a hydrophilic linker), a conventional chemical synthetic method in the art can be used, and specifically, the preparation method therefor can be substantially identical to the above method for preparing the targeted nano-carrier with L-Asp as a targeting group, which is not repeated herein.

The present disclosure provides use of the targeted nano-carrier in the above technical solutions or the targeted nano-carrier prepared by the preparation method in the above technical solutions as an active targeted nano-carrier for living plants, tissues of living plants, organs of living plants, cells of living plants, in vitro cultured explants, in vitro cultured callus, in vitro cultured plant tissues or in vitro cultured plant cells. In the present disclosure, the living plants optionally include monocots or eudicot plants. In the present disclosure, the monocots optionally include plants of the order of Asparagales, Poales, Commelinales or Arecales. The plants of the order of Asparagales optionally include plants in Orchidaceae family; the plants of the order of Poales optionally include Poaceae plants, where the Poaceae plants optionally include maize, rice, wheat, sorghum, bamboo or buckwheat; the plants of the order of Commelinales optionally include Commelinaceae plants, where the Commelinaceae plants optionally include Commelina communis L. In the present disclosure, the eudicot plants include plants of the order of Asterales, Cucurbitales, Fabales, Solanales or Brassicales, where the plants of the order of Asterales optionally include plants in the Asteraceae family; the plants of the order of Cucurbitales optionally include plants in the Cucurbitaceae family; the plants of the order of Fabales optionally include plants in the Fabaceae family, where the plants in the Fabaceae family optionally include soybean or pea; the plants of the order of Solanales optionally include plants in the Solanaceae family, where the plants in the Solanaceae family include tomato, pepper or potato; the plants of the order of Brassicales optionally include plants in the Brassicaceae family, where the plants in the Brassicaceae family optionally include Arabidopsis or rape. In the present disclosure, the organs of living plants optionally include leaves, seeds or roots; the in vitro cultured plant cells optionally include in vitro cultured protoplasts, where the in vitro cultured protoplasts specifically may be prepared from leaves, hypocotyls or root tips. In the present disclosure, the targeted nano-carrier can actively target and penetrate plant cell walls and cell membranes, forming “Trojan horse”-type cellular penetration and targeting. When used for drug delivery to plants, the targeted nano-carrier in the present disclosure can reduce drug dosage and costs, have a protective effect on loaded drugs and improve drug efficacy, prolong therapeutic duration, reduce toxicity and contamination, and reduce drug resistance probability.

In the present disclosure, the targeted nano-carrier can be used alone, or mixed with a non-targeted nano-carrier. When the targeted nano-carrier is used in combination with a non-targeted nano-carrier, the targeted nano-carrier optionally accounts for more than 1% by mass of a total mass of the targeted nano-carrier and the non-targeted nano-carrier, specifically 20-80%. The non-targeted nano-carrier of the present disclosure specifically refers to a nano-carrier that is free of any modification or that is modified with a substance other than the target of the present disclosure.

The present disclosure provides a targeted drug-loaded nano-carrier, including a targeted nano-carrier and a drug encapsulated in the targeted nano-carrier, where the targeted nano-carrier is the targeted nano-carrier in the above technical solutions or the targeted nano-carrier prepared by the preparation method in the above technical solutions. In the present disclosure, drug-loading rate of the targeted drug-loaded nano-carrier is optionally 1-99%, and more optionally 30-80%. In the present disclosure, the drug optionally includes a small molecule drug or a biomacromolecule, where the biomacromolecule optionally includes nucleic acid, protein, amino acid, polypeptide, carbohydrate substance or lipid substance, and the nucleic acid specifically may be DNA or RNA; and the small molecule drug optionally includes plant hormone, water retaining agent, growth promoter, pesticide, antifreeze, anti-heat agent, anti-ultraviolet agent, fluorescein, transgenic agent or isotope-labeled compound, where the plant hormone optionally includes auxin, gibberellin, cytokinin, ethylene, jasmonic acid, brassinosteroid, strigolactone, abscisic acid (ABA) or ABA analogs (such as Pyrabactin, Quinabactin, Opabactin, AM1, AMF1α, AMF1β, AMF2α, AMF2β, AMF4 or AMC1β). In embodiments of the present disclosure, ABA is specifically described as an example. ABA is an endogenous plant hormone, and can close leaf stomata, reduce water transpiration and activate downstream stress-resistant signals when plants are stressed by water withholding, salt and so on, thus achieving an effect of drought resistance of the plants, but its high cost and instability in vitro limit its use in agricultural production. Encapsulation of ABA within the nano-carrier provided in the present disclosure can effectively enhance drought resistance effect of plants, and greatly reduce the ABA amount.

In the present disclosure, binding modes between the drug and the targeted nano-carrier optionally include hydrophilic and hydrophobic interactions, hydrogen bonds, electrostatic interactions, or chemical bonding.

The present disclosure provides a preparation method for the targeted drug-loaded nano-carrier in the above technical solution, including a following step:
mixing the targeted nano-carrier, a drug and a solvent, and subjecting the mixture to an encapsulation processing, yielding the targeted drug-loaded nano-carrier.

In the present disclosure, optionally, the targeted nano-carrier and the drug are respectively dissolved in an organic solvent, and the resulting targeted nano-carrier solution and the drug solution are mixed to render a mixed solution; the mixed solution is added dropwise into water to undergo encapsulation processing, yielding a targeted drug-loaded nanoparticle. The present disclosure has no particular limitation to types of the organic solvent for preparing the targeted nano-carrier solution and the drug solution, and they can be selected according to types of the targeted nano-carrier and the drug. In embodiments of the present disclosure, taking the targeted nano-carrier (Asp-PEG-PDPA) prepared with Asp (including D-Asp, L-Asp-A, L-Asp-N or L-Asp) as a targeting group, PDPA as a hydrophobic polymer, and PEG as a hydrophilic linker as an example, the organic solvent used is optionally tetrahydrofuran; and taking ABA as the loaded drug as an example, the organic solvent used is optionally ethanol. In the present disclosure, concentrations of the targeted nano-carrier solution and the drug solution are independently optionally 1-10 mg/mL, and more optionally 5 mg/mL. In the present disclosure, a volume ratio of the targeted nano-carrier solution to the drug solution is optionally determined by a desired drug-loading capacity of the targeted drug-loaded nano-carrier, which is not particularly limited in the present disclosure. In the present disclosure, a volume ratio of the mixed solution to water is optionally (0.2-0.5):1, and more optionally (0.3-0.4):1. In the present disclosure, the mixed solution is optionally added dropwise into water, where a volume of each drop is optionally 10 μL.

In the present disclosure, during the encapsulation processing, the targeted nano-carrier and the drug undergo self-assembly (for example, self-assembly can be conducted under hydrophilic and hydrophobic interactions, charge adsorption or the like), thus yielding the targeted drug-loaded nano-carrier. The present disclosure optionally chooses appropriate encapsulation processing conditions according to characteristics of the targeted nano-carrier and the drug. Specifically, the encapsulation processing can be conducted under stirring, sonication, electrical stimulation or heating conditions, so as to improve efficiency. In embodiments of the present disclosure, taking that Asp-PEG-PDPA is the targeted nano-carrier and ABA is the loaded drug as an example, the encapsulation processing is optionally conducted at room temperature under stirring, optionally for 2-4 h, and more optionally 3 h.

After the encapsulation processing, in the present disclosure, the resulting product system is optionally subjected to centrifugal filtration in a Millipore Amicon Ultra-4 5K centrifugal filtration device, and a supernatant with the targeted drug-loaded nano-carrier dispersed therein is collected, and stored at 4° C., where the centrifugal filtration is optionally performed at a rotational speed of 3600 rpm for optionally 16 min.

The present disclosure has no particular limitation to the method of using the targeted drug-loaded nano-carrier, and any method known to those skilled in the art can be used. In the present disclosure, the targeted drug-loaded nano-carrier is optionally used in a protective form of targeted drug-loaded nano-carrier dispersion. In the present disclosure, the targeted drug-loaded nano-carrier dispersion is optionally obtained by dispersing the targeted drug-loaded nano-carrier in a solvent, where the solvent is optionally water and/or an organic solvent, and the organic solvent optionally includes ethanol, dimethyl sulfoxide or tetrahydrofuran; and the targeted drug-loaded nano-carrier dispersion is optionally at a concentration of 0.1-10 mg/mL. In the present disclosure, the targeted drug-loaded nano-carrier dispersion is optionally applied via spraying, immersion, smearing and injecting. Taking application examples as examples, specifically, the targeted drug-loaded nano-carrier dispersion is used for immersing seeds for optionally 1-168 h; or the targeted drug-loaded nano-carrier dispersion is sprayed onto plant leaves, optionally at a dosage of 10-10000 μL/cm², and more optionally 10-500 μL/cm².

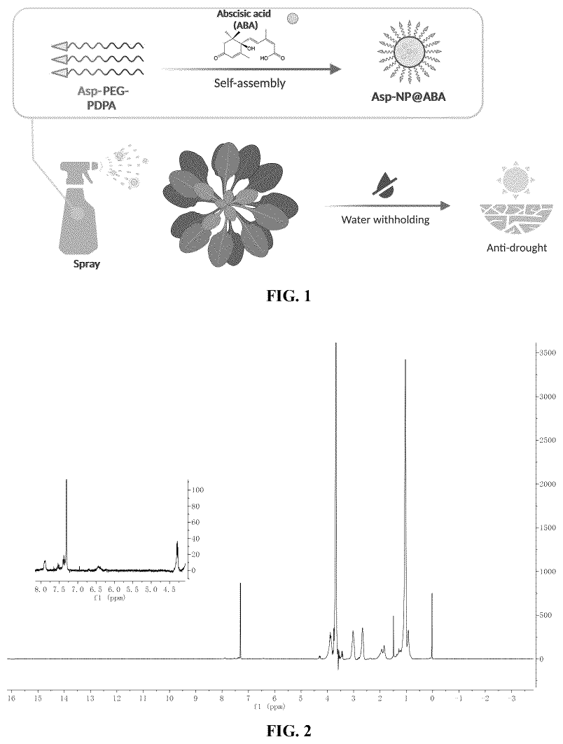

In embodiments of the present disclosure, the targeted nano-carrier (Asp-PEG-PDPA) is prepared with Asp as the targeting group, PDPA as the hydrophobic polymer, and PEG as the hydrophilic linker, and on this basis, ABA as a drug is loaded, and ABA and Asp-PEG-PDPA form the targeted drug-loaded nano-carrier (Asp-NP@ABA) via self-assembly, and the targeted drug-loaded nano-carrier is then sprayed on plant leaves to enhance drought resistance thereof. A flowchart of preparing Asp-NP and an application schematic diagram of the targeted drug-loaded nano-carrier as a drought-resistant agent obtained by loading ABA as an example are specifically shown in FIG. 1. In the present disclosure, when the targeting group Asp is D-Asp, L-Asp-A, L-Asp-N or L-Asp, respectively, micelles Asp-NP formed from various corresponding targeted nano-carriers via self-assembly are respectively designated as D-Asp-NP, A-Asp-NP, N-Asp-NP and L-Asp-NP, and targeted drug-loaded nano-carriers Asp-NP@ABA formed by loading ABA in various targeted nano-carriers via self-assembly are respectively designated as D-Asp-NP@ABA, A-Asp-NP@ABA, N-Asp-NP@ABA and L-Asp-NP@ABA.

Below, the technical solutions in the present disclosure will be described clearly and completely in conjunction with examples in the present disclosure. Obviously, only some but not all examples of the present disclosure are described. Based on the examples in the present disclosure, all of other examples obtained by those ordinarily skilled in the art without using any inventive efforts shall fall within the scope of protection of the present disclosure.

### Comparative Example 1

A reaction scheme for preparation of MeO-PEG-PDPA (compound S5) is illustrated below:

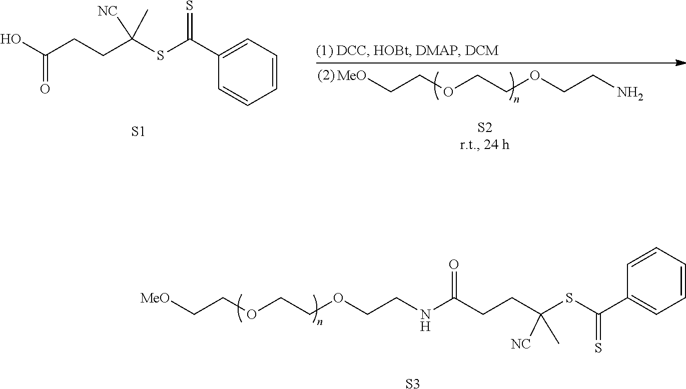

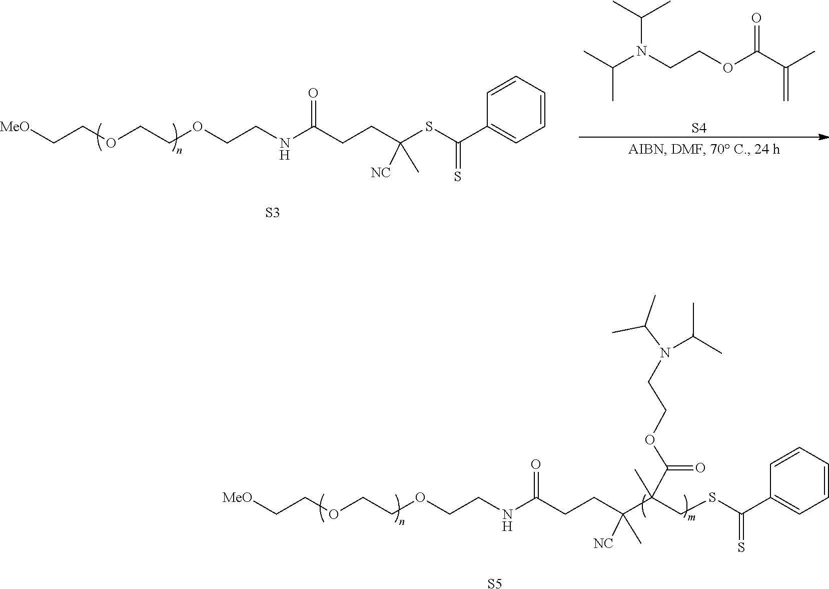

### Synthesis of MeO-PEG-CPADN (Compound S3):

Under nitrogen protection and room-temperature condition, compound S1 (4-cyano-4-(thiobenzoyl) pentanoic acid, 27.9 mg, 0.1 mmol, 1.0 equiv.), dicyclohexylcarbodiimide (DCC, 24.8 mg, 0.12 mmol, 1.2 equiv.), 1-hydroxybenzotriazole (HOBt, 16.2 mg, 0.12 mmol, 1.2 equiv.) and 4-dimethylaminopyridine (DMAP, 1.2 mg, 0.01 mmol, 0.1 equiv.) were dissolved in dichloromethane (DCM, 1 mL) and stirred for 5 min to render a mixed material; compound S2 (Amino-PEG5000-OMe, 500 mg, 0.1 mmol, 1.0 equiv.) was dissolved in DCM (5 mL), and then the mixture was added into the mixed material in one portion, and stirred and reacted for 24 h under room-temperature condition; the resulting product system was subjected to rotary evaporation to remove the solvent DCM, the resulting crude product was dissolved in EtOAc (5 mL), and filtered to remove insoluble substances, and a filtrate was concentrated to render compound S3, which was subjected to next reaction without further purification.

### Synthesis of MeO-PEG-PDPA (Compound S5):

Compound S4 (533.3 mg, 2.5 mmol, 25 equiv.) and azobisisobutyronitrile (AIBN, 1.6 mg, 0.01 mmol, 0.1 equiv.) were dissolved in N,N-dimethylformamide (DMF, 5 mL), and under nitrogen protection, the product compound S3 from the previous step was added into the resulting mixed solution, followed by stirring and reaction at 70° C. for 24 h; after the reaction was ended, the resulting product system was cooled to room temperature, and then purified in a dialysis bag, where specifically, anhydrous ethanol was used as a dialysate for dialysis for 24 h first, then the dialysate was changed into ultrapure water for dialysis for 24 h, and then a material in the dialysis bag was collected and freeze-dried to obtain the compound S5, MeO-PEG-PDPA (specifically, MeO-5kPEG-4kPDPA), as a pink amorphous solid.

### Example 1

A reaction scheme for preparation of Asp-PEG-PDPA (compound S9) with L-Asp as a targeting group is illustrated below:

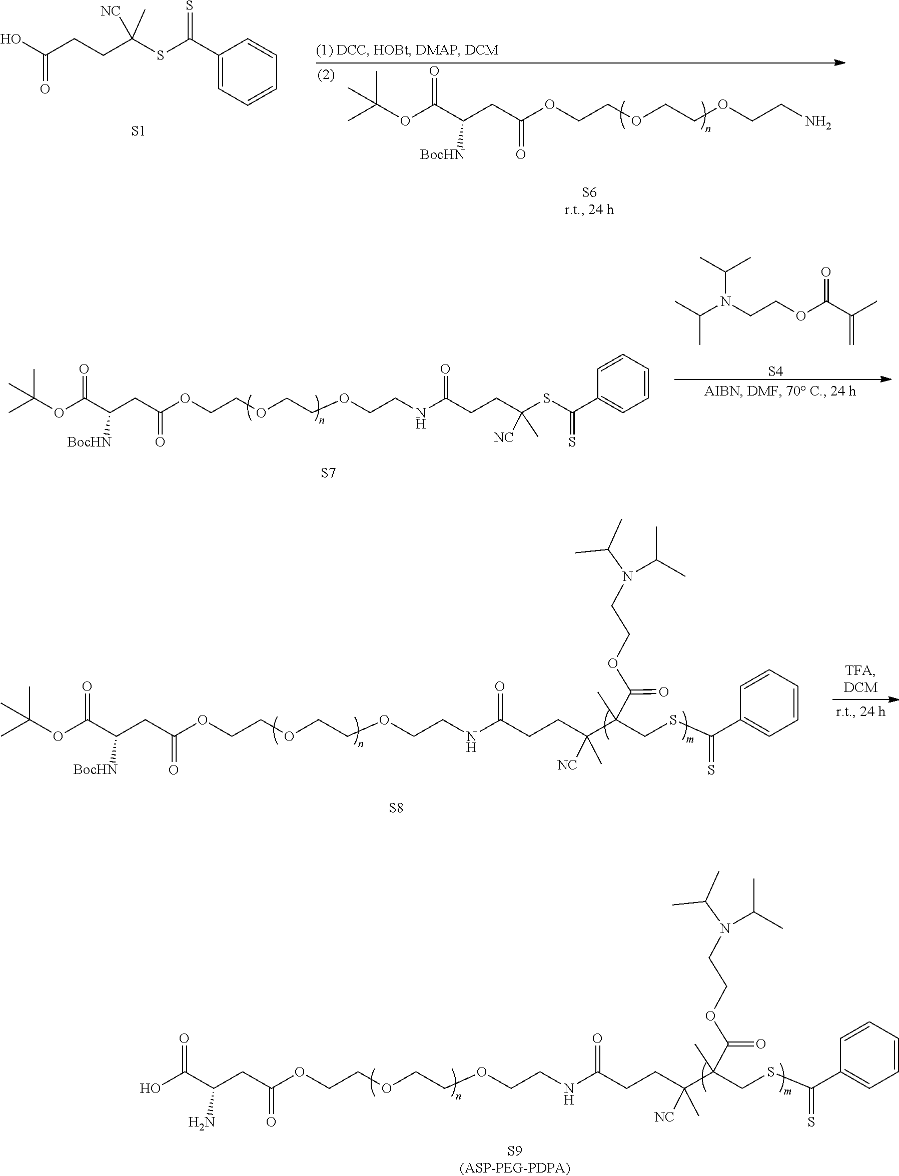

Under nitrogen protection and room-temperature condition, compound S1 (27.9 mg, 0.1 mmol, 1.0 equiv.), DCC (24.8 mg, 0.12 mmol, 1.2 equiv.), 1-hydroxybenzotriazole (HOBt, 16.2 mg, 0.12 mmol, 1.2 equiv.) and 4-dimethylaminopyridine (DMAP, 1.2 mg, 0.01 mmol, 0.1 equiv.) were dissolved in dichloromethane (DCM, 1 mL) and stirred for 5 min to render a mixed material; compound S6 (Amino-PEG5000-Boc-Asp-OtBu, 500 mg, 0.1 mmol, 1.0 equiv.) was dissolved in DCM (5 mL), and then the mixture was added into the mixed material in one portion, and stirred and reacted for 24 h under room-temperature condition; the resulting product system was subjected to rotary evaporation to remove the solvent DCM, the resulting crude product was dissolved in ethyl acetate (EtOAc, 5 mL), and filtered to remove insoluble substances, and a filtrate was concentrated to render compound S7, which was subjected to next reaction without further purification.

Compound S4 (2-(diisopropylamino)ethyl methacrylate, 533.3 mg, 2.5 mmol, 25 equiv.) and AIBN (1.6 mg, 0.01 mmol, 0.1 equiv.) were dissolved in DMF (5 mL), and under nitrogen protection, compound S7 was added into the resulting mixed solution, followed by stirring and reaction at 70° C. for 24 h; after the reaction was ended, the resulting product system was cooled to room temperature, and then purified in a dialysis bag, where specifically, anhydrous ethanol was used as a dialysate for dialysis for 24 h first, then the dialysate was changed into ultrapure water for dialysis for 24 h, and then a material in the dialysis bag was collected and freeze-dried to obtain compound S8 as a pink amorphous solid.

Under nitrogen protection, compound S8 was dissolved in a mixed solution of 5 mL of DCM and 5 mL of trifluoroacetic acid (TFA), and the resulting mixed material was stirred and reacted under a room-temperature condition for 24 h; the resulting product system was subjected to rotary evaporation to remove the solvents DCM and TFA, the resulting crude product was dissolved in EtOAc (5 mL), and then purified in a dialysis bag, where specifically, firstly, anhydrous ethanol was used as a dialysate for dialysis for 24 h (with five dialysate changes), then the dialysate was changed into ultrapure water for dialysis for 24 h (with five dialysate changes), and then a material in the dialysis bag was collected and freeze-dried to render the compound S9, as a pink amorphous solid.

With reference to the above method, Asp-PEG-PDPA with D-Asp, L-Asp-A and L-Asp-N respectively as the targeting group was prepared.

The compound S9 prepared in Example 1 was subjected to nuclear magnetic resonance characterization, and result is shown in FIG. 2. It can be determined from FIG. 2 that the product prepared in Example 1 was Asp-PEG-PDPA.

Asp-PEG-PDPA powder (the targeting group was D-Asp, L-Asp-A, L-Asp-N or L-Asp) prepared in Example 1 was made into micelle Asp-NP (specifically, D-Asp-NP, A-Asp-NP, N-Asp-NP and L-Asp-NP), followed by transmission electron microscope characterization, specifically as follows: dissolving the Asp-PEG-PDPA powder (1 mg, 0.625 mmol) in tetrahydrofuran (0.2 mL), so as to render Asp-PEG-PDPA solution; adding ultrapure water (1 mL) into a reaction vial equipped with a rotor, adding the Asp-PEG-PDPA solution into the reaction vial dropwise at 10 μL per drop, and after completing the dropwise addition, stirring the mixture under a room-temperature condition for 3 h, with all operations being conducted in a fume hood; afterwards, subjecting the resulting system to centrifugal filtration in a Millipore Amicon Ultra-4 5K centrifugal filtration device (3600 rpm, 16 min), and collecting and storing a supernatant (specifically, a liquid in an inner tube of the centrifugal filtration device) with Asp-NP dispersed therein at 4° C.

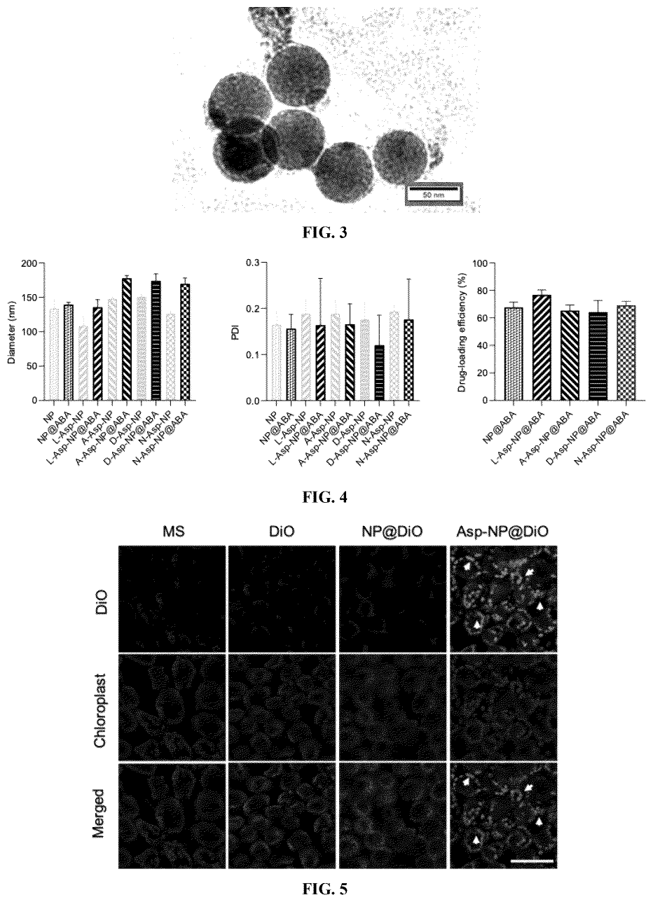

FIG. 3 is a transmission electron microscope image (scale bar being 50 nm) of L-Asp-NP in Example 1. As can be seen from FIG. 3, Asp-NP nanoparticles had a uniform size, with a particle size being around 100 nm.

### Example 2

The Asp-PEG-PDPA powder (1 mg, 0.625 mmol; the targeting group being D-Asp, L-Asp-A, L-Asp-N or L-Asp) prepared in Example 1 was dissolved in tetrahydrofuran (0.2 mL) to render Asp-PEG-PDPA solution; abscisic acid (ABA, 5 mg, 18.9 mmol) was dissolved in ethanol (1 mL) to render an ABA solution; 0.2 mL of the Asp-PEG-PDPA solution (containing 1 mg of Asp-PEG-PDPA) was mixed with a volume of ABA solution, such that the ABA accounted for 40% by mass of a total mass of Asp-PEG-PDPA and ABA, to render a mixed solution;

Into a reaction vial equipped with a rotor, ultrapure water (1 mL) was added, the mixed solution was added into the reaction vial dropwise at 10 μL per drop, and after completing the dropwise addition, the mixture was stirred under a room-temperature condition for 3 h, with all operations being conducted in a fume hood; after the reaction was ended, the resulting system was subjected to centrifugal filtration in a Millipore Amicon Ultra-4 5K centrifugal filtration device (3600 rpm, 16 min), and a supernatant (specifically, a liquid in an inner tube of the centrifugal filtration device) with a drug-loaded product (designated as Asp-NP@ABA, specifically D-Asp-NP@ABA, A-Asp-NP@ABA, N-Asp-NP@ABA and L-Asp-NP@ ABA) dispersed therein was collected and stored at 4° C.

### Comparative Example 2

A drug-loaded product was prepared according to the method in Example 2, except that the Asp-PEG-PDPA powder was replaced with MeO-PEG-PDPA, and the finally resulting drug-loaded product is designated as NP@ABA.

Particle size distribution and polydispersity index of NP (specifically, MeO-PEG-PDPA prepared in Comparative Example 1), NP@ABA, Asp-NP (specifically, D-Asp-NP, A-Asp-NP, N-Asp-NP and L-Asp-NP) and Asp-NP@ABA (specifically, D-Asp-NP@ABA, A-Asp-NP@ABA, N-Asp-NP@ABA and L-Asp-NP@ABA) were detected by dynamic light scattering (DLS), and results are shown in FIG. 4. As can be seen from FIG. 4, a particle size of the newly prepared L-Asp-NP@ABA was 135.5±4.2 nm, and PDI was 0.164±0.038 (after being left for 12 months, the particle size of L-Asp-NP@ABA was 139.4±5.9 nm, and PDI was 0.15±0.03); a particle size of the newly prepared NP@ABA was 139.53±1.21 nm, and PDI was 0.156±0.012 (after being left for 9 months, the particle size of NP@ABA was 136.6±13.1 nm, and PDI was 0.32±0.08); a particle size of the newly prepared A-Asp-NP@ABA was 177.6±1.5 nm, and PDI was 0.17±0.01; a particle size of the newly prepared D-Asp-NP@ABA was 173.9±3.8 nm, and PDI was 0.12±0.02; and a particle size of the newly prepared N-Asp-NP@ABA was 169.7±3.1 nm, and PDI was 0.18±0.03.

The drug-loading rate of Asp-NP@ABA and NP@ABA were measured, and results are shown in FIG. 4. As can be seen from FIG. 4, the drug-loading rate of L-Asp-NP@ABA was 72.07±1.92%, the drug-loading rate of A-Asp-NP@ABA was 65.3±1.4%, the drug-loading rate of D-Asp-NP@ABA was 64.2±2.8%, and the drug-loading rate of N-Asp-NP@ABA was 69.1±1.0%; and the drug-loading rate of NP@ABA was 60.09±2.79%.

In the following application examples, unless otherwise specified, the Asp targeting group refers to L-Asp; and other configurations of Asp targeting groups are explicitly labeled when used.

### Application Example 1 Asp-NP can Efficiently Penetrate Cell Walls and Membranes of Plant Tissues

Asp-NP@DiO was prepared according to the method in Example 2, except that ABA was replaced with 3,3′-dioctadecyloxacarbocyanine perchlorate (DiO; CAS: 34215-57-1).

NP@DiO was prepared according to the above method, except that Asp-PEG-PDPA was replaced with MEO-PEG-PDPA.

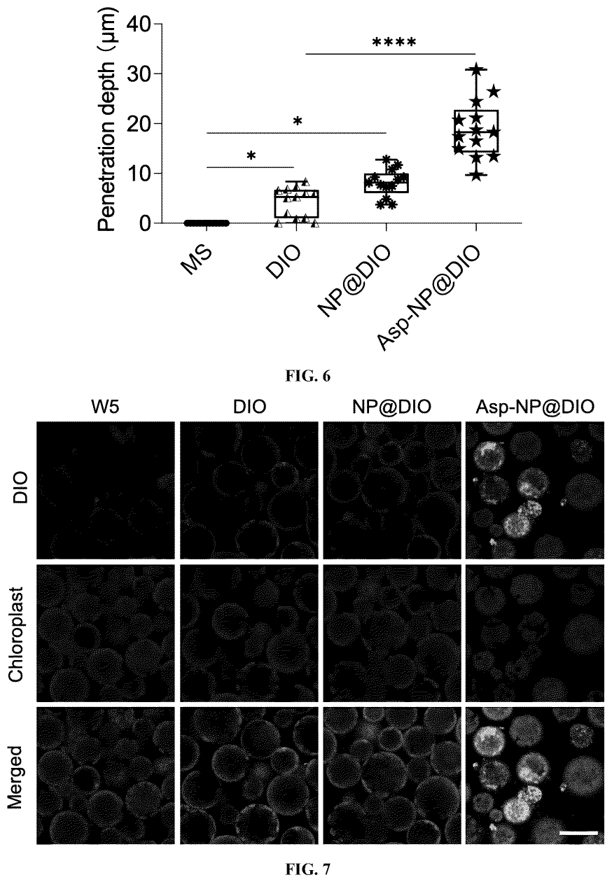

Asp-NP@DiO was dispersed in ultrapure water to obtain Asp-NP@DiO dispersion at a concentration of 31.8 μM; the Asp-NP@DiO dispersion was sprayed onto 14-day-old Arabidopsis leaves at approximately 60 μL per plant; after 36 h, penetration depths of DiO fluorescence signals from leaf surfaces vertically downward into mesophyll tissues were tracked using a confocal laser microscope based on 3D layer-scanning function thereof, and MS, DiO and NP@DiO control groups were set, with specific results shown in FIG. 5 (scale bar being 50 μm) and FIG. 6. FIG. 5 illustrates results observed at 20 μm by the confocal microscope. From the observed DiO signals in FIG. 5, it can be seen that at this depth of 20 μm, only Asp-NP can penetrate and bring DiO into this tissue depth. Moreover, it can be seen from overlay image that the Asp-NP@DiO group showed strong co-localization of chloroplasts (red particles) and DiO signals, indicating that Asp-NP@DiO was likely to have penetrated cell walls and cell membranes into cells. Each point in FIG. 6 represents one replicate, using four Arabidopsises in total. It can be seen from FIG. 6 that Asp-NP@DiO achieved the deepest tissue penetration in the sprayed leaves, confirming strong tissue penetration capability of Asp-NP within a short period of time after spraying.

The Asp-NP@DiO dispersion and isolated Arabidopsis mesophyll protoplasts were co-cultured for 4 h; after replacing the medium with fresh W5 medium and culturing for 20 h, subcellular localization of DiO fluorescence signals from protoplasmic membrane into cells was tracked using the confocal laser microscope. W5 buffer, DiO and NP@DiO control groups were set. Specific results are shown in FIG. 7 (scale bar being 50 μm).

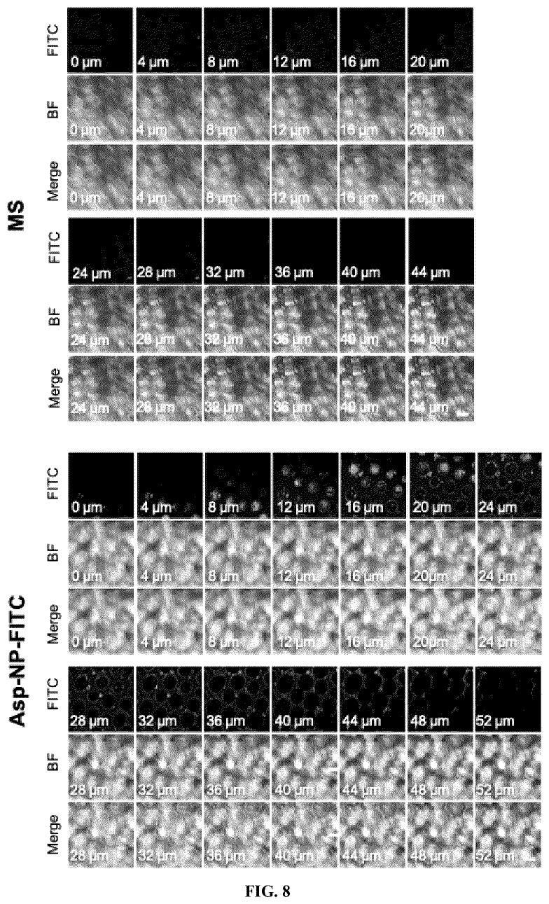

Asp-NP was labeled with fluorescein isothiocyanate (FITC) to obtain Asp-NP-FITC. The Asp-NP-FITC was dispersed in ultrapure water to obtain Asp-NP-FITC dispersion at a concentration of 12.5 μM; the Asp-NP-FITC dispersion was sprayed onto Commelina communis leaves, approximately 10 μL per leaf; after 6 h, penetration depths of FITC fluorescence signals from Commelina communis leaf surfaces vertically downward into mesophyll tissue were tracked using the confocal laser microscope based on 3D layer-scanning function thereof, and MS group was set as control, with specific results shown in FIG. 8 (scale bar being 50 μm). FIG. 8 illustrates results at various depths observed by the confocal laser microscope. As can be seen from the observed FITC signals in FIG. 8, Asp-NP can penetrate and bring FITC to a tissue depth of approximately 50 μm. Since Commelina communis leaf epidermis lacks stomata, this experiment confirmed that the targeted nano-carrier penetrated the cell walls rather than relying on stomatal entry for delivery of molecules of interest.

By combining results of FIG. 5, FIG. 6, FIG. 7 and FIG. 8, it is further demonstrated that the Asp-NP prepared in the present disclosure can efficiently penetrate tissues, cell walls and cell membranes of different plants, to deliver molecules of interest into living plant tissues and cells.

### Application Example 2 Asp-NP@ABA can Efficiently Enter Plants Via Seed Coats and Roots and Achieve Delivery

Wild-type (WT) Arabidopsis seeds were put in 24-well plates, with 20 seeds per well; Asp-NP@ABA (specifically D-Asp-NP@ABA, A-Asp-NP@ABA, N-Asp-NP@ABA or L-Asp-NP@ABA) was diluted in ½ MS (pH=6.7) liquid medium, to render Asp-NP@ABA dispersions separately with varying targeting groups (at ABA concentration of 0.1 μM), each well was added with the respective Asp-NP@ABA dispersion (400 μL) for incubation, with six replicate wells per concentration, germination was conducted in a growth chamber (16 h light/8 h dark), and germination ratios were counted every 12 h over 7 consecutive days. MS, ABA, NP, Asp-NP (specifically D-Asp-NP, A-Asp-NP, N-Asp-NP or L-Asp-NP) and NP@ABA were simultaneously set as controls.

FIG. 9 is a comparison graph of germination ratios of Arabidopsis seeds with different treatments. As can be seen from FIG. 9, Asp-NP@ABA having varying targeting groups can more effectively enter seed tissues than ABA, and ABA delivered into the seed tissues by Asp-NP can more remarkably delay the seed germination.

Asp-NP-FITC was dispersed in ½ MS medium, such that a concentration of the Asp-NP-FITC was 12.5 μM, so as to render a medium containing Asp-NP-FITC; then, roots of 7-day-old Arabidopsis seedlings, roots of 4-day-old soybean seedlings and roots of 4-day-old maize seedlings were immersed in the medium containing Asp-NP-FITC, respectively, and depths of penetration of Asp-NP-FITC into root tissues of various plants were observed at different time points. The MS group was simultaneously set as control.

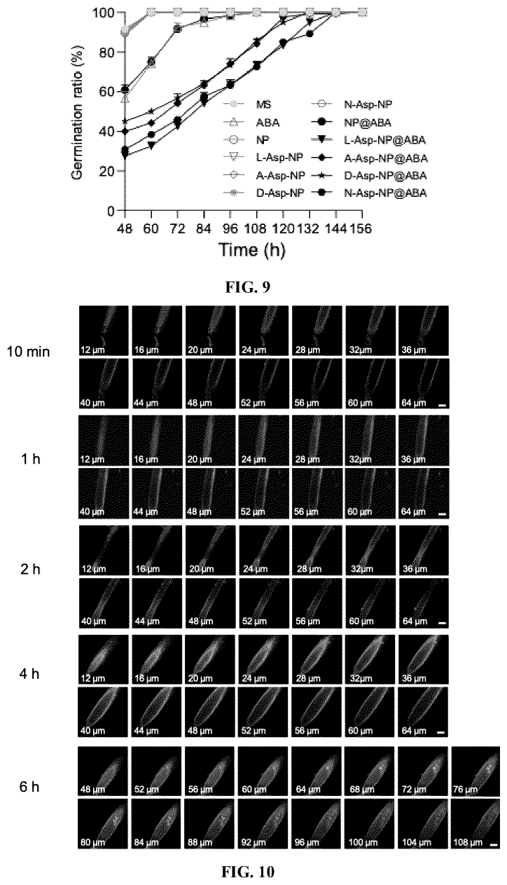

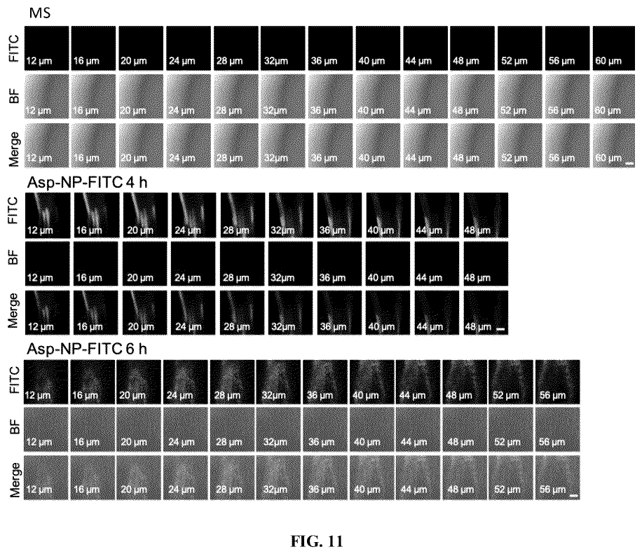

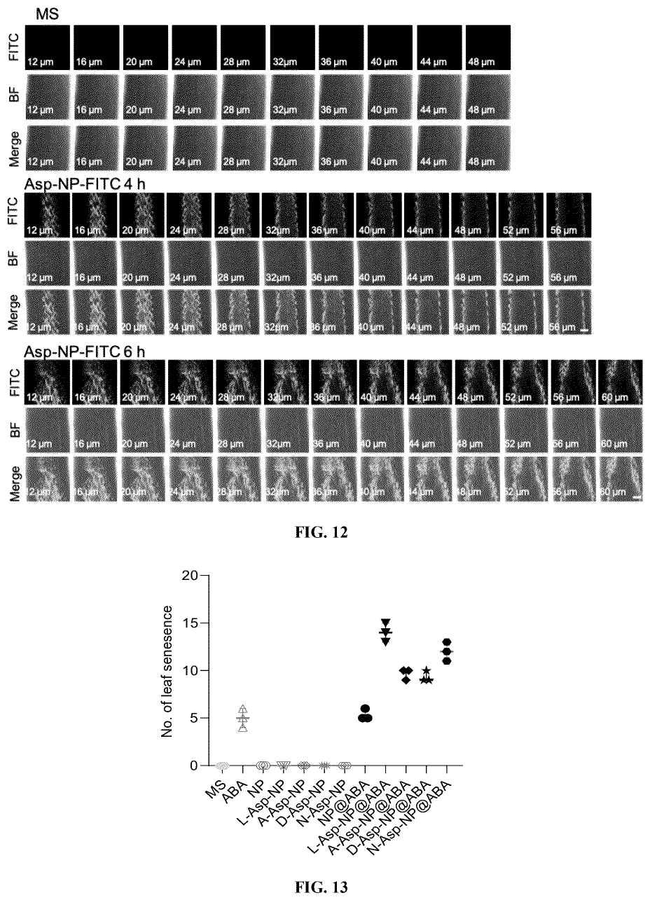

FIG. 10 shows comparison images of different penetration depths of Asp-NP-FITC into Arabidopsis root tissues at different time points (scale bar being 100 μm). FIG. 11 shows comparison images of different penetration depths of Asp-NP-FITC into soybean root tissues after 4 h and 6 h (scale bar being 100 μm). FIG. 12 shows comparison images of different penetration depths of Asp-NP-FITC into maize root tissues after 4 h and 6 h (scale bar being 100 μm). It can be seen respectively from FIG. 10, FIG. 11 and FIG. 12 that Asp-NP-FITC can efficiently enter root tissues of plants of different species via immersion.

10-day-old Arabidopsis seedlings were put in a hydroponic system, with only roots immersed in the Asp-NP@ABA dispersion (the Asp-NP@ABA used was specifically D-Asp-NP@ABA, A-Asp-NP@ABA, N-Asp-NP@ABA or L-Asp-NP@ABA, at ABA concentration of 2.5 μM) diluted with a ½ MS (pH=6.7) liquid medium. Each seedling was immersed in 1.8 mL, with 10 replicates in each experiment, the seedlings were placed in a growth chamber for germination (16 h light/8 h dark), and the number of senescent and yellowing leaves was counted every 12 h over 2 consecutive days. MS, ABA, NP, Asp-NP (specifically D-Asp-NP, A-Asp-NP, N-Asp-NP or L-Asp-NP) and NP@ABA were simultaneously set as controls.

FIG. 13 is a statistical chart of the number of ABA-induced leaf senescence and yellowing after Arabidopsis root uptake with different treatments in the hydroponic system. As can be seen from FIG. 13, compared with other treatments, Asp-NP@ABA having varying targeting groups can more efficiently deliver ABA into plant tissues via roots, ABA delivery into cells can cause leaf senescence and yellowing. Therefore, a larger number of yellowing leaves indicates higher efficiency of ABA delivery by corresponding Asp-NP.

### Application Example 3 Asp-NP@ABA Confers Drought Resistance to Model Plants

Asp-NP@ABA was dispersed in ultrapure water to obtain Asp-NP@ABA dispersion, with a concentration of ABA in the Asp-NP@ABA dispersion being 10 μM; the Asp-NP@ABA dispersion was sprayed onto Arabidopsis leaves (3 weeks old), approximately 60 μL per seedling, watering was discontinued after the spraying, seedlings grew in a growth chamber (8 h light at 22° C./16 h dark at 19.8° C.), and plant growth status was recorded daily.

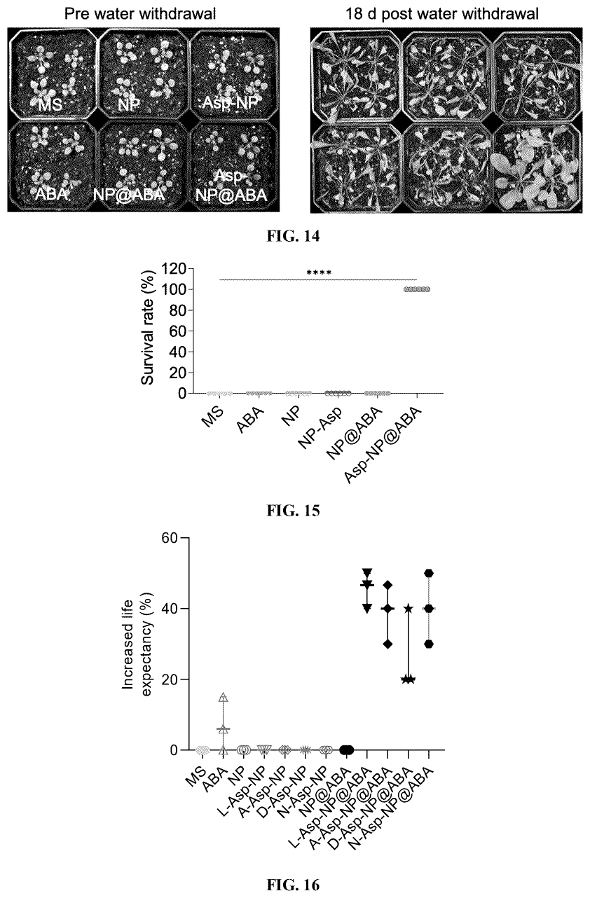

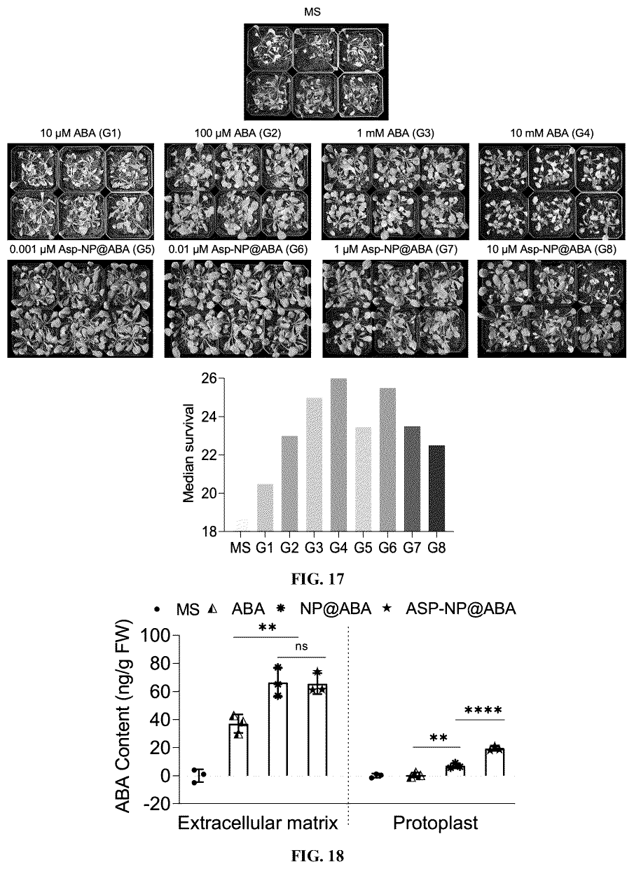

FIG. 14 shows comparison images of extended survival periods of Arabidopsis seedlings under water withholding conditions after the seedlings were sprayed with different treatments. As can be seen from FIG. 14, Asp-NP@ABA treatment group still maintained good vitality even after 18 days of water withholding compared with other groups, demonstrating that Asp-NP@ABA treatment can extend the survival periods of various plants. FIG. 15 is comparison chart of survival rates of Arabidopsis seedlings after being sprayed with different treatments. Specifically, the Arabidopsis seedlings were subjected to spraying with different treatments and maintained under water withholding conditions for the first 11 days, followed by single re-watering on the 12^{th} day, and survival rates were assessed on the 13^{th} day, further validating the efficacy of Asp-NP@ABA in enhancing plant drought resistance. It can be seen from FIG. 15 that the survival rate of the Arabidopsis plants in the Asp-NP@ABA treatment group was 100%, while the survival rates in other treatment groups were 0%. FIG. 16 is a percent scatter plot of extended survival periods of Arabidopsis seedlings under water withholding conditions after they were subjected to spraying with different treatments, relative to those in the MS treatment group (4 parallel tests were set, Asp-NP@ABA used in the present test was specifically D-Asp-NP@ABA, A-Asp-NP@ABA, N-Asp-NP@ABA or L-Asp-NP@ABA, and Asp-NP used was specifically D-Asp-NP, A-Asp-NP, N-Asp-NP or L-Asp-NP). As can be seen from FIG. 16, the survival periods of the plants after being treated with Asp-NP@ABA having different targeting groups were extended by an average of 57%. FIG. 17 shows results of minimum effective concentrations of Asp-NP@ABA determined through survival period assessment under water withholding conditions (with outcomes of ABA treatment as references). As can be seen from FIG. 17, under identical water withholding conditions, Asp-NP@ABA can reduce the abscisic acid concentration to between 10⁻⁵ and 10⁻⁶ of ABA.

Further research found that after the Arabidopsis leaf surfaces were sprayed with Asp-NP@ABA, significant ABA accumulation was detected in protoplasts and extracellular matrix (i.e., cell walls and intercellular spaces) of leaves using high performance liquid chromatography-mass spectrometry. FIG. 18 is a comparison chart of ABA content in protoplast and extracellular matrix 24 h after spraying NP@ABA and Asp-NP@ABA onto the Arabidopsis leaves. As can be seen from FIG. 18, both NP@ABA and Asp-NP@ABA can effectively transport ABA into cell walls, to make ABA to be accumulated in extracellular regions. However, ABA accumulation status in protoplasts extracted from the same leaf was different, where more ABA was accumulated in protoplast when Asp-NP@ABA was sprayed, which indicates that when the same amount of ABA was loaded into the cell walls by the nano-carrier, targeted modification of Asp can efficiently cross the cell membranes and enter the protoplast.

### Application Example 4 Asp-NP@ABA Confers Drought Resistance in Monocots or Eudicot Plants

Asp-NP@ABA was dispersed in ultrapure water to render Asp-NP@ABA dispersion, where a concentration of ABA in the Asp-NP@ABA dispersion was 10 μM; the Asp-NP@ABA dispersion at the concentration of 10 μM was sprayed onto soybean leaves (5 weeks old), approximately 600-800 μL per seedling, watering was discontinued after the spraying, seedlings grew in a growth chamber (8 h light at 22° C./16 h dark at 19.8° C.), and plant growth status was recorded daily.

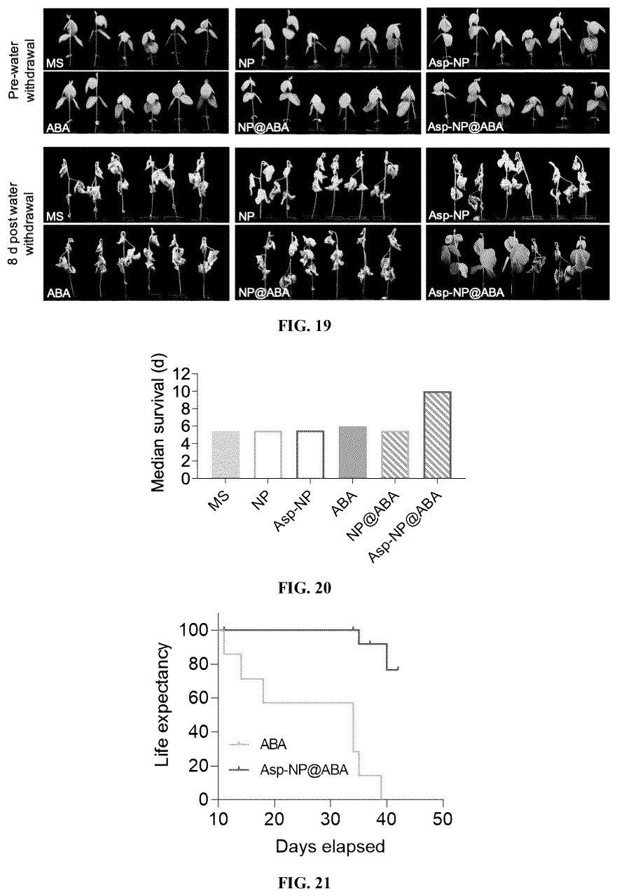

FIG. 19 shows comparison images of extended soybean survival periods under water withholding conditions after they were sprayed with different treatments. As can be seen from FIG. 19, the Asp-NP@ABA treatment group still maintained good vitality even after 8 days of water withholding compared with other groups, demonstrating that Asp-NP@ABA treatment can enhance drought resistance of various soybean plants. FIG. 20 is a median plot of extended soybean survival periods under water withholding conditions after they were sprayed with different treatments. It can be seen from FIG. 19 and FIG. 20 that after soybean plants were treated with Asp-NP@ABA, the median of the survival periods was extended by 50% on average.

Moreover, Asp-NP@ABA was dispersed in ultrapure water to obtain Asp-NP@ABA dispersion, with a concentration of ABA in the Asp-NP@ABA dispersion being 50 μM; the Asp-NP@ABA dispersion at the concentration of 50 μM was sprayed onto maize leaves (10 weeks old), approximately 7-8 mL per seedling, watering was discontinued after the spraying, seedlings grew outdoors, and plant growth status was recorded daily.

FIG. 21 is a comparison graph of extended maize survival periods under water withholding conditions after the maizes were sprayed with different treatments. As can be seen from FIG. 21, the Asp-NP@ABA treatment group still maintained good vitality even after 34 days of water withholding compared with the ABA group, demonstrating that Asp-NP@ABA treatment can enhance the drought resistance of various maize plants.

### Application Example 5

Asp-PEG-PDPA powder prepared in Example 1 was mixed with a non-targeted polymer (i.e., MeO-PEG-PDPA) to render a mixed powder, where a mass content of the Asp-PEG-PDPA powder in the mixed powder was 20%, 40%, 60%, 80% or 100%, respectively; the mixed powder (1 mg) was dissolved in tetrahydrofuran (0.2 mL) to render a mixed solution; abscisic acid (ABA, 5 mg, 18.9 mmol) was dissolved in ethanol (1 mL) to render an ABA solution; 0.2 mL of the mixed solution was mixed with a volume of the ABA solution, so that ABA accounted for 40% by mass of a total mass of the mixed powder and ABA (i.e., Asp-PEG-PDPA+MeO-PEG-PDPA+ABA), so as to obtain a ABA-containing mixed solution; into a reaction vial equipped with a rotor, ultrapure water (1 mL) was added, the ABA-containing mixed solution was added into the reaction vial dropwise at 10 μL per drop, and after completing the dropwise addition, the mixture was stirred under a room-temperature condition for 3 h, with all operations being conducted in a fume hood; after the reaction was ended, the resulting system was subjected to centrifugal filtration in a Millipore Amicon Ultra-4 5K centrifugal filtration device (3600 rpm, 16 min), and a supernatant (specifically, a liquid in an inner tube of the centrifugal filtration device) with a drug-loaded product dispersed therein was collected and stored at 4° C.

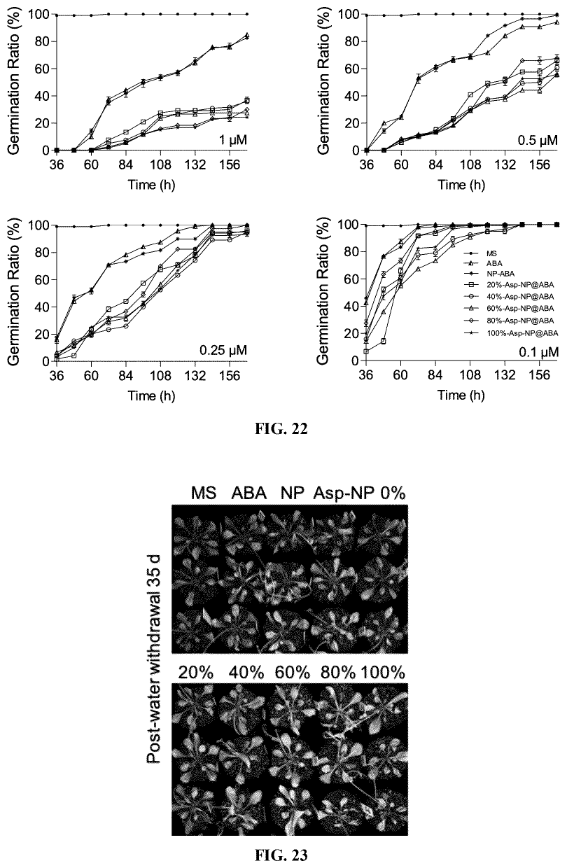

The drug-loaded products were tested for performance according to the methods in Application Example 2 and Application Example 3, and results are shown in FIG. 22 and FIG. 23. It can be seen from FIG. 22 and FIG. 23 that under identical ABA concentration condition, the drug-loaded products added with the non-targeted polymer (i.e., MeO-PEG-PDPA) at different ratios were not statistically different in the effect of in inhibiting the seed germination and the effect in extending the survival period of Arabidopsis under water withholding conditions.

It can be seen from the above application examples that the nanodelivery carrier provided by the present disclosure has at least following beneficial effects:

1) convenient operation: the loaded drug can be directly dispersed in the solvent and sprayed on surfaces of various different plants, and also can be applied via immersion, smearing or injection methods; 2) wide application range: the nanodelivery carrier is highly compatible with types of loaded drugs, has high drug-loading rate, and can be used for different plants, varieties and tissues and organs; 3) extremely high delivery efficiency: the concentration of abscisic acid can be reduced to between 10⁻⁵ and 10⁻⁶ under the condition in Application Example 3; 4) long action period: the survival period of the plants under the water withholding conditions in Application Example 3 was extended by 57%, the survival rate was 100%, while the survival rate of other treatment groups was 0%; 5) stable property: the particle size was still stable and unchanged after 12 months; and 6) controllable costs, simple technical route, and large-scale production.

Therefore, the targeted nano-carrier provided by the present disclosure has the potential of wide application, and particularly, the characteristics thereof enable excellent advantages in extremely rough and extremely harsh environments, such as scientific expedition, arid land reclamation, and extraterrestrial exploration.

The above-mentioned are merely optional embodiments of the present disclosure, and it should be indicated that those ordinarily skilled in the art still could make improvements and modifications, without departing from the principle of the present disclosure, and all of these improvements and modifications should also be considered as the scope of protection of the present disclosure.

### INDUSTRIAL APPLICABILITY

The targeted nano-carrier provided by the present disclosure includes a nano-carrier and a target chemically bonded on the nano-carrier, where the nano-carrier is a nanoparticle formed from an organic polymer or an inorganic material, and the target is aspartic acid or an aspartic acid derivative. The targeted nano-carrier provided by the present disclosure can actively penetrate plant cell walls and cell membranes, is suitable for drug delivery for living plants, tissues etc., can reduce drug dosage and costs, have a protective effect on loaded drugs and improve drug efficacy, prolong therapeutic duration, reduce toxicity and contamination, reduce drug resistance probability, and boast excellent industrial applicability.

1. A targeted nano-carrier, comprising a nano-carrier and a target chemically bonded on the nano-carrier, wherein the nano-carrier is a nano-particle formed from an organic polymer or an inorganic material, and the target is aspartic acid or an aspartic acid derivative.

2. The targeted nano-carrier according to claim 1, wherein a targeting group provided by the aspartic acid has any of structures shown in Formulas I-IV:

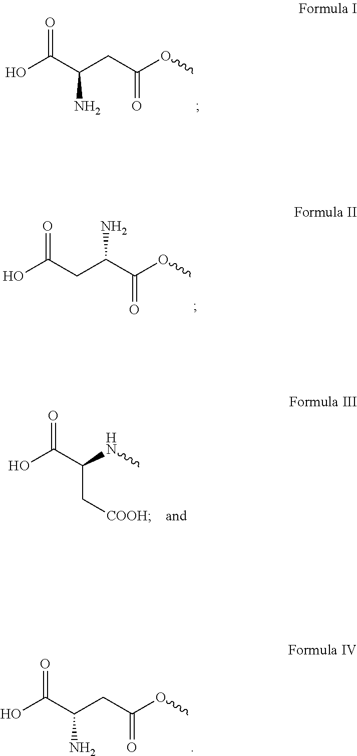

3. The targeted nano-carrier according to claim 1, wherein the nano-carrier has a particle size of 10-1000 nm.

4. The targeted nano-carrier according to claim 1, wherein a number-average molecular weight of the organic polymer is 3-50 kDa.

5. The targeted nano-carrier according to claim 1, wherein the organic polymer comprises a hydrophobic polymer and a hydrophilic linker covalently linked to the hydrophobic polymer, and the hydrophilic linker is chemically bonded to the target.

6. A preparation method for the targeted nano-carrier according to claim 1, comprising a following step:
modifying the target on the nano-carrier via chemical bonding in the presence of a solvent, so as to yield the targeted nano-carrier.

7. Use of the targeted nano-carrier according to claim 1 as an active targeted nano-carrier for living plants, tissues of living plants, organs of living plants, cells of living plants, in vitro cultured explants, in vitro cultured callus, in vitro cultured plant tissues or in vitro cultured plant cells.

8. A targeted drug-loaded nano-carrier, comprising a targeted nano-carrier and a drug encapsulated in the targeted nano-carrier, wherein the targeted nano-carrier is the targeted nano-carrier according to claim 1.

9. The targeted drug-loaded nano-carrier according to claim 8, wherein the drug comprises a small molecule drug or a biomacromolecule, and a drug-loading rate of the targeted drug-loaded nano-carrier is 1-99%.

10. A preparation method for the targeted drug-loaded nano-carrier according to claim 8, comprising a following step:
mixing the targeted nano-carrier, the drug and a solvent, and subjecting a mixture to an encapsulation processing, to yield the targeted drug-loaded nano-carrier.
